(19) United States
(12) Patent Application Publication
(10) Pub. No.: US 2022/0395607 A1
(43) Pub. Date: Dec. 15, 2022

(54) SWELLABLE ANTIMICROBIAL FIBRE

(71) Applicant: IO-CYTE LIMITED, Leeds West Yorkshire (GB)

(72) Inventors: Christopher Ochayi AGBOH, Leeds West Yorkshire (GB); David FARRAR, Leeds West Yorkshire (GB)

(21) Appl. No.: 17/771,276

(22) PCT Filed: Oct. 22, 2020
(86) PCT No.: PCT/GB2020/052663

§ 371 Date: Apr. 22, 2022

(30) Foreign Application Priority Data
Oct. 24, 2019 (GB) 1915426.9

(51) Int. Cl. A61L 15/44; A61L 15/22; A61K 33/18

(52) U.S. Cl. CPC A61L 15/44; A61L 15/225; A61K 33/18; A61L 2300/106

(57) ABSTRACT

A swellable biocompatible material and method of making the same. The material comprises a water-absorbing swellable polymer infused with povidone-iodine and a water-soluble control compound configured to control a release of iodine form the material. Example swellable polymers includes polysaccharides and hydrocolloid forming compounds.

### FIELD OF THE INVENTION

The present invention relates to a swellable polymer fibre incorporating an antimicrobial and a method of producing the same and in a particular, although not exclusively, to a swellable polymer fibre incorporating an antimicrobial agent for use as a wound dressing.

### BACKGROUND ART

A variety of different dressings have been developed for treating many different types of wounds from grazes and cuts to more serious and problematic wounds such as burns or ulcers. In particular, these latter types of wound tend to produce significant quantities of exudate which more conventional pads and bandages cannot absorb.

Wound dressings may be formed from gauzes, films, woven or non-woven fabrics and swellable materials including hydrocolloids, alginates, hydrogels, polysaccharides. Such wound dressings may be natural or synthetic and are designed specifically for their biocompatibility.

For major wounds such as burns or ulcers it can be advantageous to prevent infection by introducing an antimicrobial agent to the wound. This may be applied directly before a fresh dressing is applied or more recently the antimicrobial may be incorporated into the dressing which is then placed directly onto the wound. The therapeutic and antimicrobial properties of iodine have been known for centuries with iodine-rich plants being used in the preparation of topical pastes to reduce pain and to help with wound healing. Iodophors are complexes of iodine and a solubilizing agent or carrier. The carrier also functions to control dissociation of the iodine to provide a sustained release when incorporated in a wound dressing and present on a wound in contact with wound exudate. A particular iodophor that has found more recent medical application is povidone-iodine (PVP-I) which is a complex salt of polyvinylpyrrolidone with triiodide ions. The antimicrobial activity of an iodophor, present within a wound dressing, is dependant on the amount of ‘free’ iodine, alternatively termed ‘available’ iodine that can be released into or onto the wound. This available iodine may be identified quantitatively via iodometry.

WO 2013/140362 A1 discloses a polymeric composite material having antimicrobial and biodegradable properties. The material is used to form medical devices having antiseptic action that is formed from a matrix of alginate and PVP-I. The composite material is used for producing films, micro-capsules and suture threads from which iodine may be released.

EP 0532275 B1 describes a wound dressing having an anhydrous water-soluble gel formed from a polysaccharide or cellulosic polymer together with a humectant. The dressing may also comprise a medicament or additive such as chlorohexidine, a silver compound or an antimicrobial such as PVP-I.

US 2011/0171284 A2 describes a medical dressing for wound healing including a sucrose, PVP-I and a gelling agent sufficient to thicken the composition to control release of sucrose and iodine to the wound.

WO 2013/078998 A1 describes a slow-release ophthalmic composition containing PVP-I to treat acute ophthalmic infections. The composition includes a pharmaceutically acceptable excipient (e.g. water) and PVP-I formed in microspheres with sodium alginate.

U.S. Pat. No. 6,897,349 B2 describes methods and compositions for making a silver-containing antimicrobial hydrophilic material suited for use as wound dressings to control and manage extrudate drainage.

US 2014/0356454 A1 describes an antimicrobial wound dressing derived from gel-forming fibres such as cellulose or alginate fibres having silver ions uniformly incorporated amongst the matrix.

However, antimicrobial wound dressings are required offering enhanced exudate management and sustained and controlled release of the pharmaceutically active agent.

### SUMMARY OF INVENTION

It is an objective of the present invention to provide a pharmaceutically active fibre, filament, yarn or a material suitable as a wound dressing exhibiting moisture absorbing characteristics whilst providing a sustained and controlled release of the antimicrobial agent. It is a further objective to provide a fibrous, non-woven felt, woven or knitted material having the desired physical and mechanical characteristics for the treatment of a variety of different types of wound from minor cuts and grazes to more serious forms such as burns and ulcers.

The objectives are achieved by providing a fibre, yarn, multi filament and/or material exhibiting enhanced moisture absorbing qualities together with a controlled and sustained release of iodine (as an antimicrobial agent). In particular, the present material when positioned at a wound are effective to achieve a desired moisture vapour transmission rate from the wound and through the material, a desired physical integrity so as not to degrade when absorbing moisture and exudate, moisture retention so as to provide a hygroscopic humectant whilst also enabling convenient release or decoupling from the wound when required.

According to a first aspect of the present invention there is provided a swellable biocompatible material formed by a method of contacting a water-absorbing swellable polymer with a solution containing an antimicrobial to allow the antimicrobial to be absorbed by the swellable polymer; the solution comprising: water; povidone-iodine; and a water-soluble control compound configured to control a release of iodine from the material.

The present materials and processes are designed specifically to provide moisture absorbing, antimicrobial structures having a desired moisture vapour transmission rate (MVTR), physical integrity—so as not to degrade when exposed to exudate and in a moisture absorbed swollen configuration in addition to providing controlled and sustained antimicrobial release at the wound.

The present materials and methods may utilise a variety of different primary polymers including natural or synthetic materials. Such primary polymers are moisture absorbing swellable polymers and include for example polysaccharides or polysaccharide-based materials, hydrocolloids, biopolymers. A preferred form of primary polymer is a water-soluble polymer. Such a polymer may be a polysaccharide alginate.

Optionally, the substrate may comprise a non-water soluble synthetic polymer materials such as polyester, a polyamide, a cellulosic, an acrylic or a biopolymer.

The present fibre and methods may utilise additional compounds to further control the release of iodine from the fibre. Such additional components may include secondary compounds (that are preferably water-soluble but may be non-water soluble) such as polyethers, alkyl ethers, glycols such as propylene glycol (PG), polyols or a compound having a C—O—C linkage such as polypropylene glycol (PPG) or polyethylene glycol (PEG).

In particular, it is preferred that the water-soluble control compound is polar or at least partially polar. It is hypothesised that the water-soluble control compound may interact via electrostatics and/or structural conformation so as to at least partially inhibit disassociation of the iodine from the PVP complex. Accordingly, the iodine is inhibited from uncontrolled or free release from the material by interaction of the iodine and/or the iodine complex with the water-soluble control compound.

Optionally, the swellable polymer may comprise: a polysaccharide; a polysaccharide-based material; a hydrocolloid forming compound.

Optionally, the water-absorbing swellable polymer may comprise any one or a combination of: an alginate; chitosan; chitin; pectin; carboxymethyl cellulose; hydroxypropyl methylcellulose; gellan; konjac; psyllium or synthetic polymers such as polyesters, polyamides, polyacrylonitriles.

More preferably, the water-soluble control compound comprises propylene glycol and/or polyethylene glycol, wherein a concentration of the water-soluble control compound is such so as to provide the desired sustained release of iodine from the material when exposed to moisture/exudate at the wound. Such a concentration within the final material (suitable for use as a wound dressing) may be in the range 0.5 to 40 wt %, 0.5 to 30 wt %, 0.5 to 25 wt % or 1.0 to 20 wt % based on a total weight of the material.

Preferably, the solution further comprises a polar organic solvent. The polar organic solvent is advantageous as a substitute to reduce the amount of water to form the aqueous solution. This has been found to facilitate drying of the processed material to remove excess liquid. Incorporating a polar organic solvent within the solution with which the present material is contacted, is further beneficial to increase the softness of the final material. Such a property is advantageous for use as a wound dressing as will be appreciated. Optionally, the polar organic solvent comprises any one or a combination of an aldehyde, a ketone, an alcohol, an acetal or a compound with a hydroxyl group or a carbonyl group. More preferably, the polar organic solvent may comprise acetone and/or isopropanol.

According to a further aspect of the present invention there is provided a swellable biocompatible material comprising: a water-absorbing swellable polymer; povidone-iodine; and a water-soluble control compound configured to control a release of the iodine from the swellable polymer.

Optionally, the material may comprise the povidone-iodine at 0.5 to 40 wt %, 0.5 to 30 wt %, 0.5 to 25 wt % or 1.0 to 20 wt % based on a total weight of the material.

According to a further aspect of the present invention there is provided a method of forming a swellable biocompatible material comprising: providing a solution comprising: water; povidone-iodine; and a water-soluble control compound configured to control a release of iodine from the material, contacting a water-absorbing swellable polymer with the solution to enable at least the povidone-iodine to be absorbed by the swellable polymer; and removing the water and/or allowing the swellable polymer to dry.

Optionally, the step of contacting a swellable polymer with the solution comprises soaking, dunking, immersing, dipping or dyeing the polymer in the solution containing the PVP-I. This contact between the swellable material and the solution may be undertaken batch-wise or may be a continuous process using, for example, a rotating drum, a conveyer system, moving spray heads, mangling and the like as will be appreciated. Such systems may be automated via the use of electronic control utilising sensors, programmable logic controllers, electronic control boards, a computer and the like running software and appropriate user interfaces to provide local or remote control and status monitoring.

Optionally, the method may comprise using a conveyor system wherein the swellable polymer is moved or conveyed relative to at least one spray nozzle or head to spray the solution onto the swellable polymer; or the at least one spray nozzle or head is moved or conveyed relative to the swellable polymer to spray the solution onto the swellable polymer. Such a system may comprise multiple spray nozzles or heads to apply the solution to the swellable polymer in parallel or in-series. As will be appreciated, multiple spraying applications is configured to change the concentration of the PVP-I within the material as desired. Preferably, the material comprises ‘free’ PVP-I at a concentration of 0.5 wt. % to 10 wt. %, 0.5 wt. % to 8 wt. %, 0.5 wt. % to 6 wt. %, or 1 wt. % to 4 wt. % based on a total weight of the final material. However, the present moisture-absorbing material may comprise higher concentrations of PVP-I of the order of up to 20 wt. %. Such concentration may be advantageous for certain applications where there is a high risk of infection and a need for high bacterial control.

Optionally, the step of contacting the swellable polymer with the solution comprises contacting said polymer with said solution within a pressure vessel and applying a pressure to the solution to facilitate the absorption of the povidone-iodine into the swellable polymer.

Optionally, the step of contacting the swellable polymer with the solution comprises immersing the polymer material evenly into the solution under slight tension. This avoids deformation and moving the material out of the solution. This is followed by mangling under controlled pressure to facilitate the absorption of the povidone-iodine into the swellable polymer.

Optionally, the present material may be in a variety of different forms such as fibre-based, sliver or roving. Optionally, the present material may be a non-woven, a woven or a knitted material. Preferably, the present material is a felt-based material formed from multifilament yarn used to create the component fibres.

Importantly, the present fibre and material, once manufactured, comprises a desired moisture content. This avoids the resultant fibres agglomerating which is particularly important for a multi filament-based material. Additionally, it is important to provide a resultant material that comprises a generally uniform structure devoid of cracks or undesired large internal cavities or voids otherwise associated with a material that is allowed to dry completely. A material having a desired moisture content facilitates use as a wound dressing for placement onto a wound and to achieve the desired physical and mechanical characteristics such as the desired moisture vapour transmission rate, exudate absorption etc. Optionally, the present fibre and material comprises a moisture content in the range 5 to 60%, 10 to 60%, 15 to 55%, 20 to 50%, 25 to 50%, 30 to 50%, 30 to 45% or 35 to 40% moisture. The moisture content may be determined by any suitable method. For example, the moisture content may be determined by subtracting the dry weight of the fibre or material from the appropriately moistened fibre or material and then dividing this difference (moisture content) by the total weight of fully moistened fibre/material. The values of moisture content reported herein therefore are relative moisture wt % ranges of the amount of liquid within the moistened material.

The present fibre and material may comprise a moistening liquid being any one or a combination of water, a water-based solution, an organic liquid, an organic solution, acetone, isopropanol.

Optionally, the present fibre and/or material may comprise at least one additional or further antimicrobial agent. The further antimicrobial agent may comprise silver, a silver ion or a silver containing compound. Optionally, the further antimicrobial agent comprises a metal species being one or a combination of the set of Zn, Cu, Ti, Pt, Pd, Bi, Sn, Sb.

Preferably, the method further comprises compressing, squeezing or pressing the moistened substrate to expel excess moisture prior to allowing the moistened substrate to dry. Compressing or pressing the moistened substrate is advantageous to drive impregnation of the fibres with the iodine complex. Additionally, this step facilitates uniform distribution of the iodine complex over the surface of the fibres and optionally into the core or inner region of the fibre body.

According to a further aspect of the present material there is provided a wound dressing comprising the material as described and claimed herein. The present material may comprise a uniform concentration of the antimicrobial both in the plane of the material (laterally: lengthwise and widthwise) and through the depth or thickness of the material (perpendicular to the plane of the material) between respective first and second contact faces. Optionally, the present material may comprise a graduated or non-uniform concentration of the antimicrobial both in the lateral direction or through the thickness or depth of the material. For example, a first contact face may comprise a higher concentration of PVP-I relative to a second surface. Such a configuration may provide a material offering different levels of antimicrobial activity via placement at a wound in one or two orientations i.e., front face or reverse face contact.

According to a further aspect of the present invention there is provided a material as described and claimed herein being any one of: a nasal packing material; a dental packing material; a suture; or a seton. Optionally the present material when used as a wound dressing or packing material is non-woven. Optionally the present material when used as a suture or a seton is fibrous.

### BRIEF DESCRIPTION OF DRAWINGS

A specific implementation of the present subject matter will now be described, by way of example only, and with reference to the accompanying drawings in which:

FIG. 1 illustrates schematically apparatus for introducing povidone-iodine (PVP-I) to a swellable biocompatible material including a perforated drum and a solution dispensing container;

FIG. 2 illustrates schematically a variation of the apparatus of FIG. 1 further comprising a pump located between the perforated drum and the pressure vessel;

FIG. 3 illustrates schematically selected components for the introduction of PVP-I to the swellable material using a pressurized reaction vessel;

FIG. 4 illustrates schematically apparatus for introducing PVP-I to the swellable polymer comprising a conveyer system to laterally translate the swellable material adjacent spray heads adapted to spray the PVP-I solution onto the conveyed swellable material; and

FIG. 5 illustrates schematically apparatus for introducing povidone-iodine (PVP-I) to a swellable biocompatible material or non-swellable material comprising an impregnation trough containing a solution, dipping guide rollers and a pair of mangling rollers;

FIG. 6 illustrates schematically apparatus for introducing povidone-iodine (PVP-I) to a swellable biocompatible material or non-swellable material comprising an impregnation trough containing a solution, dipping guide rollers and a pair of mangling rollers, according to a further specific implementation;

FIG. 7 illustrates schematically apparatus for introducing povidone-iodine (PVP-I) to a swellable biocompatible material or non-swellable material comprising an impregnation trough containing a solution, dipping guide rollers and a pair of mangling rollers, according to a further specific implementation;

FIG. 8 illustrates schematically apparatus for introducing povidone-iodine (PVP-I) to a swellable biocompatible material or non-swellable material comprising an impregnation trough containing a solution, dipping guide rollers and a pair of mangling rollers, and a drying enclosure according to a further specific implementation;

FIG. 9 is a graph of zone of inhibition versus available iodine present within the swellable material using bacteria types Staphylococcus and Klebsiella.

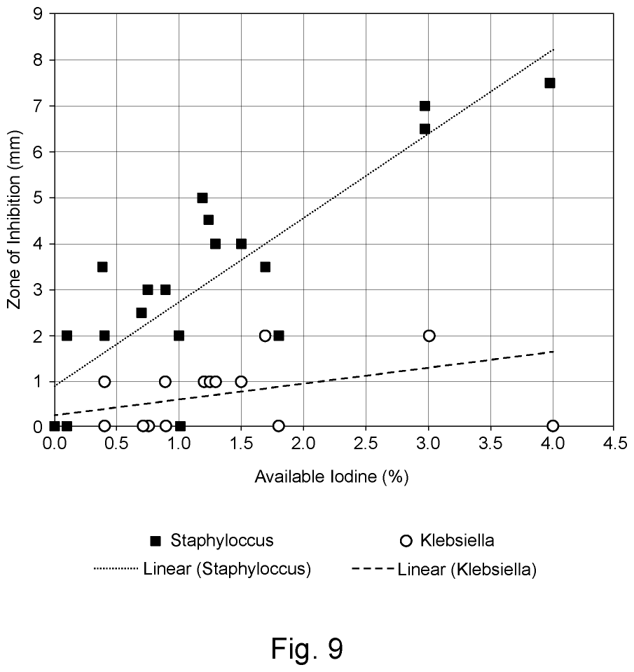

### DETAILED DESCRIPTION OF PREFERRED EMBODIMENTS OF THE INVENTION

The present antimicrobial swellable fibre is suitable for the manufacture of a non-woven felt-like material, a woven or knitted material that, in turn, may be utilised as a wound dressing for the treatment of a variety of different types of wound from cuts and grazes to more serious burns, ulcers and the like where exudate management is critical. The present material is conveniently formed from an infusing, soaking, immersing or dyeing process in which the fibres, sliver, roving, a woven, a non-woven or a knitted material is introduced to a generally aqueous solution containing povidone-iodine (PVP-I). The present materials and processes provide processing parameters to produce highly swellable materials incorporating ‘available’ iodine within the fibre/yarn core and at the fibre/yarn surface. The iodine is in a form of an iodine complex and included at a concentration level sufficient to provide antimicrobial activity. Such concentration levels may be of the order of greater than 1 wt % ‘free’ iodine within a total weight of fibre. The present materials and processes are specifically designed to provide a material having a desired moisture vapour transmission rate (MVTR), physical integrity—so as not to degrade when exposed to exudate and in a moisture absorbed swollen configuration, in addition to providing controlled and sustained antimicrobial release at the wound.

The present materials and methods may utilise a variety of different primary polymers including natural or synthetic materials. Such primary polymers are moisture absorbing swellable polymers and include for example polysaccharides or polysaccharide based materials, hydrocolloids, biopolymers. Preferred forms of primary polymers are water-soluble polymers such as but not limited to a polysaccharide alginate.

The present fibre and methods may utilise additional compounds to further control the release of iodine from the fibre. Such additional components may include secondary water-soluble control compounds such as polyethers, alkyl ethers, a glycol such as propylene glycol (PG), a polyol or a compound having a C—O—C linkage such as polypropylene glycol (PPG) or polyethylene glycol (PEG).

The present examples describe procedures developed to incorporate PVP-I into alginate and similar biomaterials in various forms such as fibres, sliver, roving, non-woven, woven or knitted. The present methods all include providing a solution containing solvated PVP-I and then contacting a moisture absorbing swellable polymer with the solution to allow at least the PVP-I to be absorbed by the polymer. As will be appreciated, the resulting materials include a swellable polymer having PVP-I absorbed within the fibres of the material and also PVP-I adsorbed at the fibre surfaces. This dual presence of PVP-I provides at least two mechanisms of release. For example, the PVP-I adsorbed at the fibre surfaces may be released quickly and/or immediately as the wound dressing is brought into contact with the wound whilst absorbed PVP-I (incorporated at the fibre/yarn core) may be adapted for slower more sustained release over time. The water-soluble control compound also present within the fibres/yarn of the material provides further control of the release of ‘free’ iodine to promote a sustained release.

### EXAMPLES

Various example methods may be used in the manufacture of a swellable biocompatible material according to the present invention. Such methods may in i) batchwise processing using for example an open or pressurised chamber, or ii) continuous manufacture for example using a rotatable drum at ambient or elevated pressure or a conveyor system, or iii) batch or continuous manufacture system using an impregnation trough and mangling rollers.

Generally and with reference to Table 1 for the concentrations and amounts of materials used, the solutions were prepared using various percentages (based on the weight of biomaterial to be treated) of PVPI, water, IPA (and/or acetone), PEG (and/or PG). The required PVPI was weighed into water or water/PEG or PG and either dissolved manually or with a Greaves small high shear mixer. This was then followed by the addition of the rest of substances required and mixing until a homogeneous bulk solution was obtained for dyeing.

### Initial Example—Batchwise

The swellable moisture-absorbing material may be prepared via a simple ‘dyeing’ of the material with the PVP-I. In such a method, a vessel, chamber or tank may be used to contain the PVP-I solution. In certain examples, a polymer based non-woven felt-like material may then be dipped, placed or soaked in the solution according to a conventional dyeing process. Such a process may be undertaken at ambient pressure and temperature.

In particular, an alginate felt having the dimensions 65 cm×130 cm was folded carefully without stretching to fit into a dyeing vessel containing half of the dyeing solution. The felt now folded (e.g. 20 cm×20 cm dimension) was transferred into the vessel (e.g. a 10-litre bucket) and pressed into the solution using a paddle or any non-sticky device with a flat bottom. The second half of the solution was then poured on top of the felt and the felt pressed down gently until all the surface covered uniformly with the solution. The gentle pressing down and moving the material around in the vessel continued until all the solution was absorbed into the material-. The dyed material was taken out, placed on top of a flat polyethene black bag and gently unfolded and left under extraction system in the fume cupboard to dry for at least 4 hours. During drying, the materials was turned so that both sides were equally dried. After drying properly, the material was folded and put inside a sealed silver coated bag and stored for analysis. On occasions, portions of the dyed material were cut, left exposed in the lab without cover and observed for colour changes to determine the stability of PVI in the materials.

Various thicknesses or widths of non-wovens were produced from the single layer treated material. To produce a double layer treated felt, two single layer treated felts of the same size or a single layer treated felt folded in half was used. One side of the felt was placed on a clean flat surface and the other side of the same felt (top side) was uniformly smeared thinly with 1-3% w/w solution of high G alginate containing the same amount of PVPI and PEG or PG as used for the single layer treatment. The felt was then folded into half with the smeared faces together or if using two separate single layer treated felts, the second layer was then carefully placed symmetrically on the smeared top and a uniform pressure applied to binder the felts together. Such pressure was achieved by placing a flat plate of slightly bigger dimension and containing uniformly distributed weights on top of the combined felt and leaving it in this position for at least 2 h. At end, the felts (now combined) was removed, hung together with foldback clips (paper clamp binder clips) to prevent excessive shrinkage and dried properly under extraction system for at least 5 h.

To produce a triple layer treated felt, three equal separate layers of a treated single layer felt were used. One layer was smeared on both sides with high G alginate/PVPI/PEG solution described above, then sandwiched between the other two layers and treated as above.

For fibres, they could be dyed stuck together and continuous as would be the case for roving, but best results were obtained when the fibres were in staple form, properly separated and opened. Some fibres contained spin finish such as Tween 20 (up to 3.5%) and treated without problem. The solution preparation and material treatment were as described above for non-woven. During treatment, before and after drying, some of the staple fibres were observed to roll into little balls. But after drying, they were soft and easily opened.

TABLE 1
Incorporation of PVPI into biomaterials through ‘dyeing’ process.

| | | | | Quantity (%, g on the weight of sample or material, OWM for treatment | | | | | |
|---|---|---|---|---|---|---|---|---|---|
| | | | Amount of | | | | | IPA | |
| Example | Batch No. | Sample form | sample g | PVPI | Water | PG | PEG | (IPA:Fibre) | Acetone (Ace:Fibre) |
| 1 | P-170510-HM/ | High M-Alg.- | 120 | 6.7%, | 16.7%, | 8.3%, | — | — | (3.33:1) 400 g |
| | PVPI/PG/Dyed | psyllium staple fibres | | ~8 g | 20 g | 10 g | | | |
| | | (opened) | | | | | | | |
| 2 | P-170602-HM/ | High M-Alg.- | 120 | 3.3%, | 25%, | — | — | (3.33:1) 400 g | — |
| | PVPI/ Dyed | psyllium staple fibres | | 4 g | 30 g | | | | |
| | | (opened) | | | | | | | |
| 3 | P-170323-HM/ | High M-Alg.- | 106 | 8.5%, | 49%, | — | — | (3.77:1) 400 g | — |
| | PVPI/Dyed | psyllium staple fibres | | 9 g | ~52 g | | | | |
| | | (opened) | | | | | | | |
| 4 | P-170512-HM/ | High M-Alg.- | 89 | 10%, | 51% | — | 14.6%, | (3.37:1) 300 g | — |
| | PVPI/PEG/Dyed | psyllium staple fibres | | 8.85 | | | 13 g | | |
| | | (opened) | | | | | | | |
| 5 | AMS-14-2AP/ | High G-Alg./CMC | 68 | 10%, | 53%, | | 15%, | (3.38:1) | — |
| | PVPI/PEG/Dyed | staple fibre | | 6.8g | 36 g | | 10.2 | 230g | |
| 6 | HM-181030/ PVPI/ | High M alginate | 107 | 10% | 188% | — | 15% | (3.5:1) 375g | — |
| | PEG/Dyed/l | fibres (opened) | | 10.7 g | 201 g | | 16.05 g | | |
| 7 | HM-181030/ PVPI/ | High M alginate | 108 | 15% | 53% | — | 15% | (3.5:1) 378 g | — |
| | PEG/Dyed/2 | fibres (opened) | | 16.2 g | 57.2 g | | 16.2 g | | |
| 8 | HG-181107/PVPI/ | High G alginate | 111 | 10% | 50% | — | 15% | (4:1) 444 g | — |
| | PEG/Dyed/l | fibres (opened) | | 11.1 g | 55.5 g | | 16.6 g | | |
| 9 | HG-181107/PVPI/ | High G alginate | 111 | 15% | 50% | — | 15% | (4.2:1) 471 g | — |
| | PEG/Dyed/2 | fibres (opened) | | 16.65 g | 55.5g | | 16.65 g | | |
| 10 | P-181128 -HM/ | Single layer High M | 1.5 | 22.7% | 40.7% | | 20.7% | (3.6:1) 31 g | |
| | PVPI/PEG/Dyed | Alg./psyllium felt | | 0.34 g | 0.61g | | 0.31 g | | |
| | (XIRAMS21S) | produced by AMS | | | | | | | |
| | | from Xiros fibres | | | | | | | |
| | | (XIRAMS 21) | | | | | | | |
| 11 | XIRAMS-181204/ | XIRAMS 21 | 85 | 15.3% | 53% | — | 15.3% | (3.53:1) | — |
| | PVPI/PEG/Dyed/1 | | | 13.0 g | 45 g | | 13 g | 300 g | |
| | (XIRAMS21S) | | | | | | | | |
| 12 | XIRAMS-190819/ | XIRAMS 21 | | | | | | | |
| | PVPI/PEG/Dyed/1 | | | | | | | | |
| | (XIRAMS 2 IS) | | | | | | | | |

| | | | |
|---|---|---|---|
| 13 | XIRAMS-190819/ | XIRAMS 21 | 2 pieces of a single layer High M Alginate/psyllium felt producedby AMS from Xiros fibres |
| | PVPI/PEG/Dyed/2 | | and dyed with PVPI (XIRAMS-PVPI/PEG/Dyed/l) were taken, each smeared thinly on one |
| | (XIRAMS 21D) | | side with Alg./PVPI/PEG solution, stuck together and left under pressure for at 2 h to properly |
| | | | bind and then dried well under extraction system. |
| 14 | XIRAMS-181204/ | XIRAMS 21 | As above in example 13 except that 3 pieces of the single layer dyed felt (XIRAMS S21) were |
| | PVPI/PEG/Dyed/3 | | used and two sides of the middle layer smeared thinly with Alg./PVPI/PEG solution before |
| | (XIRAMS 2IT) | | sandwiching between the other two layers. |

TABLE 2
Some examples of fluid absorbency and retention properties
of some alginate, alginate/psyllium
fibres or felt dyed with PVPI containing PG or PEG.

| | Absorbency, g/g | | | |
|---|---|---|---|---|
| | (Retention, %) | | | |
| | Solution A | | Saline | |
| Example/Batch No. | 1 min | 30 min | 1 min | 30 min |
| 1/ | 25.06 | 33.38 | 30.68 | 30.80 |
| P-170510-HM/PVPI/PG/Dyed | (74) | (67) | (74) | (69) |
| 2/ | 26.81 | 25.80 | 29.54 | 29.69 |
| P-170602-HM/PVPI/Dyed | (74) | (75) | (87) | (84) |
| 3/ | 22.45 | 21.92 | 24.43 | 26.04 |
| P-170323-HM/PVPI/Dyed | (76) | (75) | (73) | (68) |
| 4/ | 30.52 | 25.56 | 31.72 | 28.76 |
| P-170512-HM/PVPI/PEG/Dyed | (70) | (67) | (76) | (73) |
| 5 | 20.28 | 21.95 | 25.18 | 21.38 |
| AMS-14-2AP/PVPI/PEG/Dyed | (54) | (51) | (51) | (59) |
| 6 | 31.17 | 28.17 | 30.79 | 33.92 |
| HM-181030/PVPI/PEG/Dyed/1 | (80) | (76) | (84) | (70) |
| 7 | 22.60 | 24.32 | 25.83 | 27.48 |
| HM-181030/PVPI/PEG/Dyed/2 | (85) | (78) | (82) | (88) |
| 8 | 10.75 | 20.46 | 18.99 | 22.14 |
| HG-181107/PVPI/PEG/Dyed/1 | (45) | (33) | (65) | (58) |
| 9 | 13.85 | 14.59 | 17.00 | 22.81 |
| HG-181107/PVPI/PEG/Dyed/2 | (58) | (54) | (48) | (54) |
| 10 | | | | |
| P-181128-HM/PVPI/PEG/Dyed | | | | |
| (XIRAMS 21S) | | | | |
| 11 | 23.66 | 15.74 | 12.06 | 27.55 |
| XIRAMS-181204/PVPI/PEG/Dyed/1 | (55) | (64) | (60) | (69) |
| (XIRAMS 21S) | | | | |
| 12 | | | | |
| XIRAMS-190819/PVPI/PEG/Dyed/1 | | | | |
| (XIRAMS 21S) | | | | |
| 13 | | | | |
| XIRAMS-190819/PVPI/PEG/Dyed/2 | | | | |
| (XIRAMS 21D) | | | | |
| 14 | 9.69 | 8.40 | 10.04 | 9.98 |
| XIRAMS-181204/PVPI/PEG/Dyed/3 | (36) | (31) | (66) | (60) |
| (XIRAMS 21T) | | | | |

Accordingly, the present method provides a versatile processing route for the incorporation of PVPI into fibre, yarn, sliver, roving etc -based materials (including wovens, non-wovens and knitted structures). The present processes and materials are advantageous in that:
PVPI can be dissolved homogeneously and applied to a substrate completely and easily.
the solvent(s) used are miscible to any extent with water and other additives such as diols (e.g. propylene glycol) or polyols (e.g. polyethylene glycol) that may be in the composition
the main liquid(s) used are PVPI carrier(s) only and do not react with PVPI or other chemicals in the composition
the main media are volatile solvents that are easily removed from the substrate/PVPI/additives composition or system after application by simple extraction.

The organic solvents used are preferably non-toxic alcohols, ketones, acetates or mixtures. Such solvents may be incorporated at 2:1 to 5:1 ratio relative to the substrate. Similarly, as described herein, the dyeing process could also be achieved by spraying the substrates from a pressurised vessel or can filled with a solution of PVPI/PEG (or PG)/IPA/water.

As shown in Table 2, based on the materials of the initial examples, the process improved significantly the fibre and felt strength, absorbency and iodine retention across all examples especially those containing high G alginates.

TABLE 3
Examples of iodine contents of some alginate, alginate/psyllium
fibres or felt dyed with PVPI containing PG or PEG.

| | | Iodine Content | |
|---|---|---|---|
| | Amount of | | In-house |
| | PVPI | | titration % |
| | used | | Available |
| | (on the | External | iodine |
| | weight of | results | (after |
| | fibre or | Total | samples |
| | felt, owf) | Iodine | extracted |
| Example/Batch No. | (%) | (mg/Kg) | overnight) |
| Commercia product, Inadine ™ | n/a | 1600 | 1.0 |
| 1/ | 6.7 | Not | 0.1 |
| P-170510-HM/PVPI/PG/Dyed | | Tested | |
| 2/ | 3.3 | 1200 | 0.8 |
| P-170602-HM/PVPI/Dyed | | | |
| 3/ | 8.5 | Not | 1.2 |
| P-170323-HM/PVPI/Dyed | | Tested | |
| 4/ | 10 | 2100 | 1.2 |
| P-170512-HM/PVPI/PEG/Dyed | | | |
| 5 | 10 | 1200 | 1.3 |
| AMS-14-2AP/PVPI/PEG/Dyed | | | |
| 6 | 10 | Not | 0.8 |
| HM-181030/PVPI/PEG/Dyed/1 | | Tested | |
| 7 | 15 | Not | 2.1 |
| HM-181030/PVPI/PEG/Dyed/2 | | Tested | |
| 8 | 10 | Not | 1.5 |
| HG-181107/PVPI/PEG/Dyed/l | | Tested | |
| 9 | 15 | Not | 1.7 |
| HG-181107/PVPI/PEG/Dyed/2 | | Tested | |
| 10 | 22.7 | Not | 3.0 |
| P-181128-HM/PVPI/PEG/Dyed | | Tested | |
| (XIRAMS 21S) | | | |
| 11 | 15.3 | Not | 3.0 |
| XIRAMS-181204/PVPI/PEG/Dyed/1 | | Tested | |
| (XIRAMS 21S) | | | |
| 12 | | Not | Not |
| XIRAMS-190819/PVPI/PEG/Dyed/1 | | Tested | Tested |
| (XIRAMS 21S) | | | |
| 13 | | Not | Not |
| XIRAMS-190819/PVPI/PEG/Dyed/2 | | Tested | Tested |
| (XIRAMS 21D) | | | |
| 14 | | Not | 4.0 |
| XIRAMS-181204/PVPI/PEG/Dyed/3 | | Tested | |
| (XIRAMS 21T) | | | |

Table 3 shows the iodine contents (iodide+titratable iodine) in some of the samples that were tested. The results obtained were all encouraging as they showed comparative contents (1200-2100 mg/kg) with a commercial product, Inadine™ (1600 mg/kg). The percentage available iodine (iodine that can be titrated with sodium thiosulphate) appeared also to meet all commercial requirements. Table 3 also illustrates that the use of 3-10% PVPI in the dyeing bath achieved the commercial target of 1% available iodine in the sample.

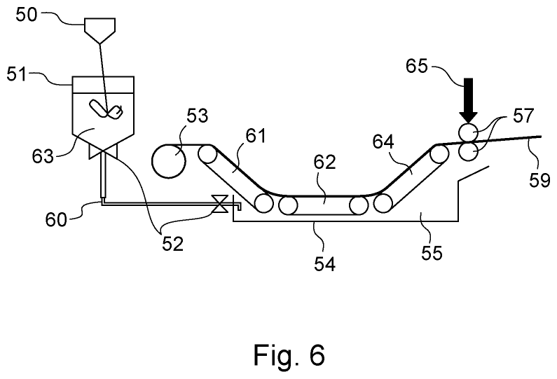

As detailed with reference to FIG. 6 and Table 4, the examples appeared to demonstrate a correlation between the amount of available iodine in the sample and its effectiveness against bacteria especially Staphylococcus. The effect on Klebsiella appeared less marked.

TABLE 4
Zone of inhibition and antimicrobial efficacy of some selected samples.

| | | % Available | | |
|---|---|---|---|---|
| | | Iodine (after | Zone of inhibition (mm) | |
| Examples | Batch no. | extract overnight) | Staphyloccus | Klebsiella |
| | HM-181024-PVP-I/PEG/1 SPUN | 0.9 | 3 | 1 |
| 4 | P-170512-HM-PVP-I/PEG DYED | 1.2 | 5 | 1 |
| 6 | AMS-14-2AP PVPI/PEG DYED | 1.3 | 4 | 1 |
| | Inadine ™ | 1.0 | 2 | 2 |
| | Blank—HM-180704-PVP (beginning) | 0.0 | 0 | 0 |
| | Alg-181030-HM PVP-I/PEG/1 (10% PVPI) DYED | 0.8 | 3 | 0 |
| 7 | Alg-181030-HMPVP-I/PEG/2 (15% PVPI) DYED | 1.3 | 4.5 | 1 |
| 8 | HG-181107-PVP-I/PEG/1 (10% PVPI) DYED | 1.5 | 4 | 1 |
| 9 | HG-181107-PVP-I/PEG/2 (15% PVPI) DYED | 1.7 | 3.5 | 2 |
| | HM-181116-PVP-I/PEG/1 SPUN | 0.4 | 2 | 0 |
| | HM/HG-180910-PVP-I/PG-10/2 SPUN | 0.7 | 2.5 | 0 |
| | HM/HG-180831-PVP-I/PG-4 SPUN | 0.9 | 3 | 0 |
| 11 | XIRAMS-181204—felt/PVP-I/PEG/DYED/1 | 3.0 | 6.5 | 2 |
| | (XIRAMS 21S—single layer) | | | |
| 14 | XIRAMS-181204—felt/PVP-I/PEG/DYED/3 | 4.0 | 7.5 | 0 |
| | (21T—triple layer) | | | |
| | P-181128-HM/PVPI/PEG/DYED (XIRAMS 21S) | 3.0 | 7 | 2 |
| | HG-181204-PVP-I/PEG SPUN | 0.4 | 3.5 | 1 |
| | Blank—Activeheal ™ (Ca/Na-Alginate) | 0.0 | 0 | 0 |
| | #23394 | | | |
| | AF-14-140730-HM (batch 1) | 0.0 | 0 | 0 |
| | HM-180207 PVPI | 1.0 | 0 | 0 |
| | HM-180212-P-PVPI | 0.1 | 0 | 0 |
| | HG-180214-PVP-I | 0.1 | 2 | 0 |
| | HG-180216-PVP-I | 1.8 | 2 | 0 |

### Production of Alginate-Iodine-Glycol Fibres and Felts

The potential controlling effects of the glycols (PG and PEG) on the ‘leaching-out’ of PVPI from fibres was investigated by incorporating non-complex iodine into fibres and felts through:
Extrusion of dopes containing dissolved iodine crystals
Dyeing fibres or non-woven with solutions containing dissolved iodine crystals.

The samples preparations were as described in example 1 for alginate-PVPI fibres except that 0.5% w/w iodine crystals (supplied by Alfa Aesar, mp 183-1860C, mol. wt (or FW253.81, density 4.930 g/cm3) were used in place of PVPI. Small amounts of PG or PEG were used to ground the iodine crystals using mortal/pestle before adding to the solution and mixing homogeneously to give very dark solutions.

Fibres produced had deep iodine colour initially, but this faded very quickly on exposure to air and even after sealing, the iodine gradually vaporised out of the fibres leaving it colourless. Addition of PG or PEG retarded evaporation but did not stop it.

### Further Examples—Batchwise

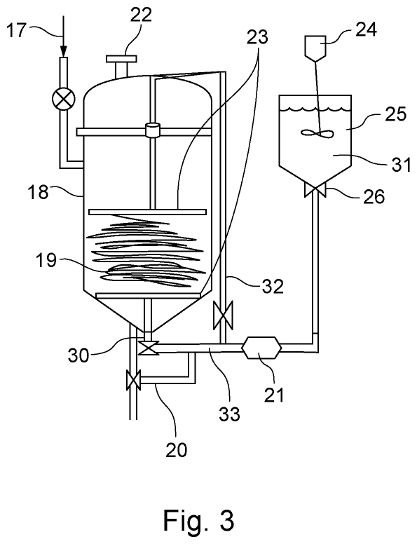

Referring to FIG. 3, a non-woven felt 19 may be combined with the PVP-I (and additives) solution 31 using a pressure vessel 18. In particular, nitrogen or compressed air may be introduced into vessel 18 via inlet 17. Vessel 18 further comprises a pressure relief valve 22 and an outlet/outlet 30 connected to drain 20 that may in turn be coupled to a return pipe system (not shown). A pump 21 drives introduction of the PVP-I solution 31 from a mixing tank 25. Solution 31 may be stirred using mixer 24. Solution 31 is pumped into the pressure vessel 18 by conduits 32, 33 and into spray heads 23 for delivery onto substrate 19. Increased pressure within vessel 18 drives the solution into the fibres of the swellable material which may then be extracted from the vessel 18 and dried.

### Further Examples—Continuous

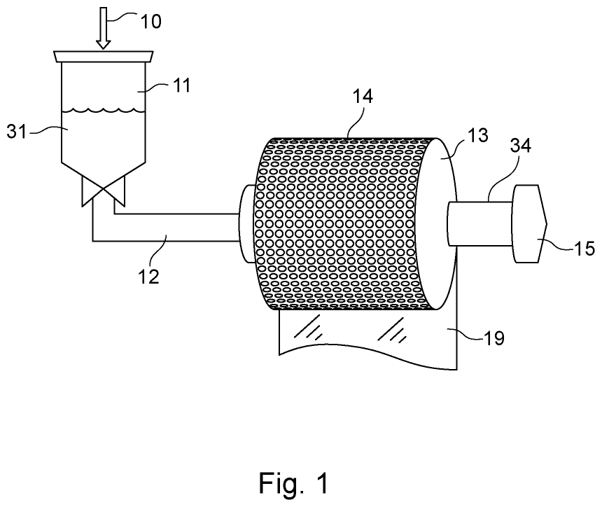

Referring to FIG. 1, a non-woven felt-like substrate may be treated with the PVP-I solution according to continuous processing in which the antimicrobial containing solution is applied or delivered to the substrate in a non-batchwise manner. Referring to FIG. 1, the non-woven substrate material 19 may be rolled onto a rotatable drum 13 mounted on axle 34 and rotatably driveable by motor 15. Drum 13 comprises perforations 14. The rotating drum assembly 13, 34, 14 is mountable within an extraction chamber or system (not shown). A connection conduit 12 provides a coupling between drum 13 and a supply tank 11 housing the PVP-I (and additives) solution 31. In operation, solution 31 is supplied to drum 13 via conduit 12. The solution 31 flows from the drum interior via perforated holes 14 onto the substrate 19 that is continuously wound around the drum external surface. Pressure may be applied to the solution 31 to force it through perforations 14 to drive absorption of the PVP-I into the swellable polymer of the substrate 19.

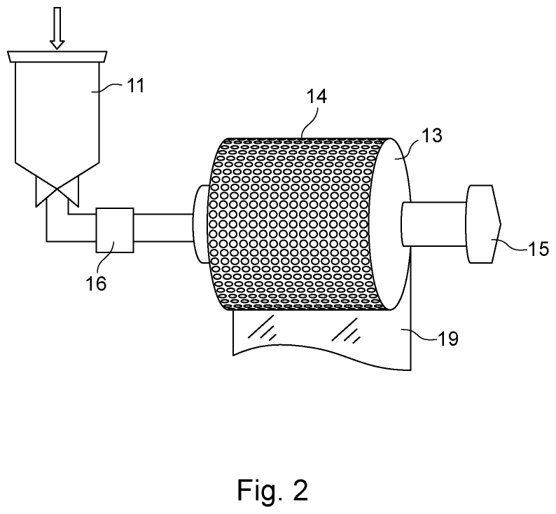

FIG. 2 illustrates a variation of the embodiment of FIG. 1 further comprising a pump 16 to further drive solution 31 under pressure into drum 13. The extent of absorption and adsorption of the PVP-I at the fibres of the swellable material may be controlled by controlling the applied pressure 10 and/or pump 16.

### Example 4—Continuous

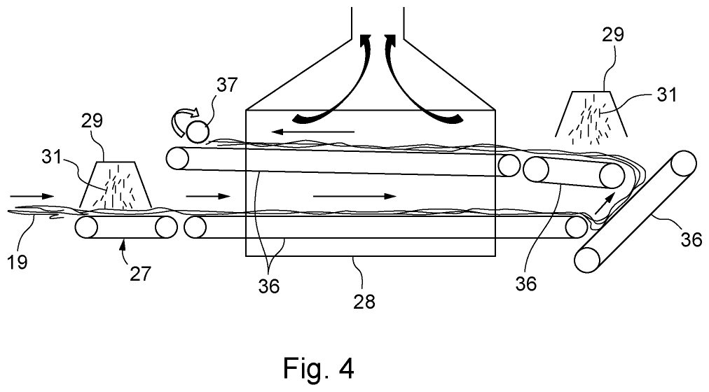

Referring to FIG. 4, the non-woven swellable material 19 may be saturated or infused with PVP-I via a conveyer system. In particular, the non-woven substrate 19 is delivered onto a conveyor system 27 that provides lateral translation of the substrate 19 below a plurality of spray heads 29 mounted at different positions relative to the various conveyers 36. At least some of the conveyors 36 pass within a fume extraction and drying chamber 28. The PVP-I (and additives) solution 31 is sprayed onto the moving substrate 19 via spray heads 29 as a continuous process. A winding roller 37 may be positioned at a terminal end of the conveyer 36 to collect the infused substrate ready for down-stream processing i.e. cutting, stamping and the like.

### Example 5—Continuous

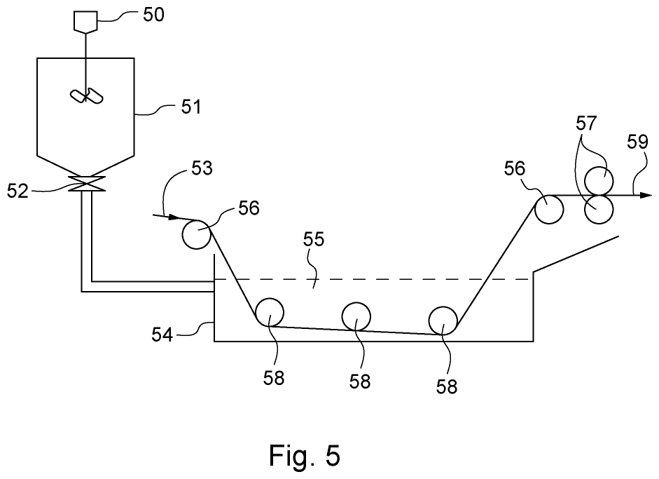

With reference to FIG. 5, the substrate 53 in form of non-woven, tow, sliver or yarn from a processing line or reel is impregnated by means of immersion rollers 58 in PVP-I solution 55 contained in a trough 54. The PVP-I laden substrate is pulled slowly through the solution and guided over the guiding rollers 56 and passed between the pair of smooth mangle rollers 57 with variable pressure system, where the PVP-I is gently pressed into the substrate and excess solution pressed out and returned to the trough. The level of solution in the trough is monitored and kept constant by replenishment from the tank 51 by opening valve 52. The mixer 50 is used to prepare the PVP-I solution and for occasional stirring when necessary. The treated substrate 59 is then passed through a drying chamber (not shown) and dried before further processing.

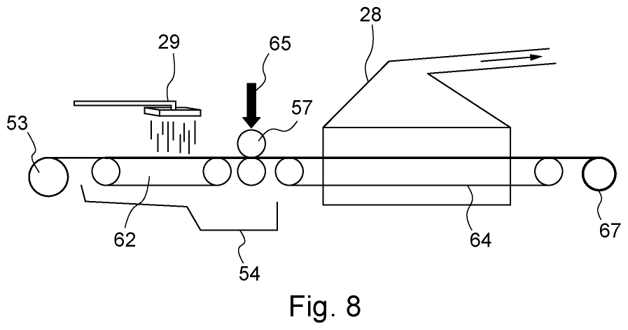

With reference to FIG. 6, the substrate 53 in form of non-woven positively driven to a delivery conveyor 61 is impregnated by means of immersion conveyor 62 in PVP-I solution 55 contained in a bath 54. The PVP-I laden substrate is gently laid on the feed conveyor 64 and moved slowly through the solution and then fed between a pair of variable speed smooth mangle rollers 57 with variable pressure system 65, where the PVP-I is gently pressed into the substrate and excess solution pressed out and returned to the impregnation bath 54. The level of solution in the trough is monitored and kept constant by replenishment from the tank 51 by opening valves 52. The mixer 50 is used to prepare the PVP-I solution and for occasional stirring when necessary. The treated substrate 59 goes through a drying chamber 28 on a second feed conveyor 64 (not shown in FIG. 6 but described referring to FIGS. 4 and 8) and dried before winding onto a reel if necessary, for further processing.

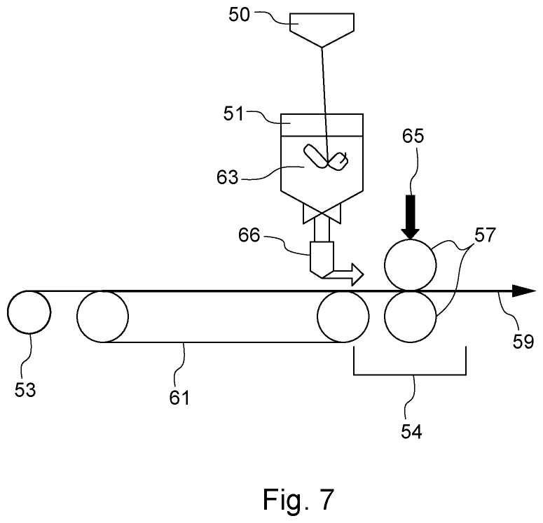

With reference to FIG. 7, the substrate positively driven to the feed conveyor 61 is fed straight into the variable speed mangle rollers under variable pressure, but just before entry into the mangles, the PVP-I solution from tank 51 is fed at a constant rate using a peristaltic pump and delivered through a suitable nozzle onto the substrate. The PVP-I laden substrate gently passes between the rollers where the PVP-I solution is pressed into the substrate and excess solution if any, pressed out into a trough 54 for reuse. As before, the treated substrate is then led onto a conveyor through a drying chamber, dried and wound onto a reel for further processing. This method is simple, avoids excessive use of conveyors and PVP-I solution and avoids also, excessive loss of PVP-I solution through evaporation.

With reference to FIG. 8, the substrate positively driven from the reel is gently laid on top of conveyor 62, where it is sprayed with PVP-I solution monitored at a controlled rate from a shower-like system 29. The PVP-I laden substrate then gently passes between the mangle rollers where the PVP-I solution is pressed into the substrate and excess solution if any, pressed out into a trough 54 for reuse. As before, the treated substrate is then led onto a conveyor through a drying chamber, dried and wound onto a reel 67 for further processing.

### Antimicrobial Activity Testing

From a preliminary investigation and as detailed in FIG. 6 and Table 4, selected of the above examples appeared to demonstrate a correlation between the amount of available iodine in the sample and its effectiveness against bacteria especially Staphylococcus. The effect on Klebsiella appeared less marked. The antimicrobial activity performance of various of the above examples were then tested further according to AATCC test method 100.

In particular, Staphylococcus aureus (ATCC 6538) and Pseudomonas aeruginosa (ATCC 9027) suspensions were prepared to 1.0×10⁶ CFUmL⁻¹ in Tryptone Soya Broth (TSB). The inoculum was enumerated by performing 10-fold dilutions in TSB and plating out the resulting suspensions onto Tryptone Soya Agar (TSA). A 1 ml aliquot of each bacterial suspension was used to inoculate control and test dressing (dimensions 4.2×4.2 cm). Test dressings were incubated for 24 hours at 37±2° C., following which, dressings were neutralised in 10 mL neutraliser. Test dressing samples were sonicated for 5 minutes to recover microorganisms from the dressing. The resultant suspensions were serially diluted and plated onto TSA to quantify viable organisms. Negative control, positive control and sterility control samples were tested simultaneously. The negative control was the same alginate base material as the test samples but without povidone iodine treatment. The positive control was a commercial carboxymethyl cellulose dressing containing silver. Samples were tested in triplicate.

### Performance Results

Staphylococcus aureus

An average of 5.61±0.27 log₁₀CFU per sample S. aureus were recovered from the negative control at 0 hours. No viable S. aureus were recovered following treatment with examples 11 to 14 (XIRAMS 21S and 21T) tested within detection limits (minimum limit of detection was 1 log). This equated to a log reduction of 4.61-5.61±0.27 log₁₀CFU per sample compared to the negative control.

TABLE 5
Antimicrobial activity results for Staphylococcus aureus

| | Log Recovery | Log Reduction |
|---|---|---|
| | (Log₁₀CFU | (Log₁₀CFU |
| Dressing | per sample ± SD) | per sample ± SD) |
| Negative control | 5.61 ± 0.27 | N/A |
| XIRAMS 21S | <1.00 | 4.61-5.61 ± 0.27 |
| XIRAMS 21T | <1.00 | 4.61-5.61 ± 0.27 |
| Positive control | <1.00 | 4.61-5.61 ± 0.27 |

Pseudomonas aeruginosa

An average of 6.12±0.05 log₁₀CFU per sample P. aeruginosa were recovered from the negative control at 0 hours. No viable P. aeruginosa were recovered following treatment with examples 11 to 14 (XIRAMS 21S and 21T) tested within detection limits (minimum limit of detection was 1 log). This equated to a log reduction of 5.12-6.12±0.05 log₁₀CFU per sample compared to the negative control.

TABLE 6
Antimicrobial activity results for Pseudomonas aeruginosa

| | Log Recovery | Log Reduction |
|---|---|---|
| | (Log₁₀CFU | (Log₁₀CFU |
| Dressing | per sample ± SD) | per sample ± SD) |
| Negative control | 6.12 ± 0.05 | N/A |
| XIRAMS 21S | <1.00 | 4.61-5.61 ± 0.27 |
| XIRAMS 21T | <1.00 | 4.61-5.61 ± 0.27 |
| Positive control | <1.00 | 4.61-5.61 ± 0.27 |

### Testing of Biofilm Disruptions—CDC Bioreactor

The performance of various of the above examples to disrupt a biofilm was tested using a CDC bioreactor. In particular, cultures of S. aureus (NCTC 8325) and P. aeruginosa (NCIMB 10434) were used to prepare single-species bacterial inoculums (1×10⁷±5×10⁶ CFUml⁻¹). The prepared S. aureus and P. aeruginosa bacterial suspensions were used to inoculate separate sterile CDC reactors containing polycarbonate coupons. The CDC reactors were incubated for 72 hours at 37° C.±2° C. in an orbital microbiological incubator to encourage biofilm growth. Following incubation, biofilm-containing coupons were washed in PBS to remove planktonic bacteria and were then treated with wound dressings (2×2 cm) for 24 hours. Negative and positive controls were tested concurrently. The negative control dressing was the same alginate base material as the test samples but without povidone iodine treatment. The positive control dressing was a commercial carboxymethyl cellulose dressing containing silver. Internal negative and positive control treatments were also included, these being PBS+1% TSB and a bleach-based product respectively. Samples were tested in triplicate.

Following treatment, coupons were transferred to 10 mL of neutraliser and the coupons were sonicated for 5 minutes to recover remaining attached microorganisms. Serial dilutions were carried out on the resultant recovery medium and total viable organisms were quantified.

### Performance Results

Staphylococcus aureus

Averages of 5.55±0.42 log₁₀CFUml⁻¹ and 5.71±0.49 log₁₀CFUml⁻¹ viable S. aureus were recovered from the internal negative control and the negative control dressing, respectively. No viable S. aureus were recovered following treatment with examples 11 to 14 (XIRAMS 21S and 21T), internal positive control and positive control dressing. This was a reduction of 5.55 log₁₀CFUml⁻¹ compared to the internal negative control.

TABLE 7
Biofilm Disruptions
(CDC Bioreactor results) for Staphylococcus aureus

| | Average Recovery | Log Reduction |
|---|---|---|
| Test Item | (Log₁₀CFUml⁻¹ ± SD) | (Log₁₀CFUml⁻¹ ± SD) |
| Internal negative control | 5.55 ± 0.42 | N/A |
| Negative control | 5.71 ± 0.49 | N/A |
| XIRAMS 21S | 0.00 ± 0.00 | 5.55 |
| XIRAMS 21T | 0.00 ± 0.00 | 5.55 |
| Internal positive control | 0.00 ± 0.00 | 5.55 |
| Positive control dressing | 0.00 ± 0.00 | 5.55 |

Pseudomonas aeruginosa

Averages of 7.09±0.06 log₁₀CFUml⁻¹ and 6.43±0.48 log₁₀CFUml⁻¹ viable P. aeruginosa were recovered from the internal negative control and the negative control dressing, respectively. No viable P. aeruginosa were recovered following treatment with examples 11 to 14 (XIRAMS 21S and 21T), internal positive control and positive control dressing. This was a reduction of 7.09 log₁₀CFUml⁻¹ compared to the internal negative control.

TABLE 7
Biofilm Disruptions
(CDC Bioreactor results) for Pseudomonas aeruginosa

| | Average Recovery | Log Reduction |
|---|---|---|
| Test Item | (Log₁₀CFUml⁻¹ ± SD) | (Log₁₀CFUml⁻¹ ± SD) |
| Internal negative control | 7.09 ± 0.06 | N/A |
| Negative control | 6.43 ± 0.48 | N/A |
| XIRAMS 21S | 0.00 ± 0.00 | 7.09 |
| XIRAMS 21T | 0.00 ± 0.00 | 7.09 |
| Internal positive control | 0.00 ± 0.00 | 7.09 |
| Positive control dressing | 0.00 ± 0.00 | 7.09 |

1. A swellable biocompatible material formed by a method of contacting a water-absorbing swellable polymer with a solution containing an antimicrobial to allow the antimicrobial to be absorbed by the swellable polymer, the solution comprising:
water;
povidone-iodine; and
a water-soluble control compound configured to control a release of iodine from the material.

2. The material as claimed in claim 1 wherein the swellable polymer comprises:
a polysaccharide;
a polysaccharide based material; or
a hydrocolloid forming compound.

3. The material as claimed in claim 1 wherein the water-absorbing swellable polymer comprises any one or a combination of:
an alginate;
chitosan;
chitin;
pectin;
carboxymethyl cellulose;
hydroxypropyl methylcellulose;
gellan;
konjac;
psyllium; or
a synthetic polymers.

4. The material as claimed in claim 1 wherein the water-soluble control compound comprises a polyether, an alkyl ether, a glycol, a polyol or a compound having a C—O—C linkage.

5. (canceled)

6. The material as claimed in claim 1 wherein the solution further comprises a polar organic solvent.

7. (canceled)

8. (canceled)

9. A swellable biocompatible material comprising:
a water-absorbing swellable polymer;
povidone-iodine; and
a water-soluble control compound configured to control a release of the iodine from the swellable polymer.

10. The material as claimed in claim 9 wherein the swellable control compound comprises any one or a combination of:
a polysaccharide;
a polysaccharide based material;
a hydrocolloid forming compound;
an alginate;
chitosan;
chitin;
pectin;
carboxymethyl cellulose;
hydroxypropyl methylcellulose;
gellan;
konjac;
psyllium; or
a synthetic polymers.

11. The material as claimed in claim 9 wherein the water-soluble control compound comprises a polyether, an alkyl ether, a glycol, propylene glycol, a polyol or a compound having a C—O—C linkage, polypropylene glycol or polyethylene glycol.

12. The material as claimed in claim 9 wherein the material comprises the water-soluble control compound in an amount of 5 to 40 wt based on a total weight of the material.

13. The material as claimed in claim 9 wherein the material comprises the povidone-iodine in an amount of 0.5 to 40 wt % based on a total weight of the material.

14. (canceled)

15. (canceled)

16. A method of forming a swellable biocompatible material comprising:
providing a solution comprising:
water;
povidone-iodine;
a water-soluble control compound configured to control a release of iodine from the material; and optionally
a polar organic solvent;
contacting a water-absorbing swellable polymer with the solution to enable at least the povidone-iodine to be absorbed by the swellable polymer; and
removing the water and/or allowing the swellable polymer to dry.

17. (canceled)

18. The method as claimed in claim 16 wherein the step of contacting the swellable polymer with the solution comprises soaking said polymer in the solution.

19. The method as claimed in claim 16 wherein the step of contacting the swellable polymer with the solution comprises spraying the solution onto the swellable polymer.

20. The method as claimed in claim 19 wherein:
the swellable polymer is moved or conveyed relative to at least one spray nozzle or head to spray the solution onto the swellable polymer; or
the at least one spray nozzle or head is moved or conveyed relative to the swellable polymer to spray the solution onto the swellable polymer.

21. The method as claimed in claim 16 wherein the step of contacting the swellable polymer with the solution comprises contacting said polymer with said solution within a pressure vessel and applying a pressure to the solution to facilitate the absorption of the povidone-iodine into the swellable polymer.

22. The method as claimed in claim 16 wherein the swellable polymer comprises any one or a combination of:
a polysaccharide;
a polysaccharide based material;
a hydrocolloid forming compound;
an alginate;
chitosan;
chitin;
pectin;
carboxymethyl cellulose;
hydroxypropyl methylcellulose;
gellan;
konjac;
psyllium; or
a synthetic polymers.

23. The method as claimed in claim 16 wherein the water-soluble control compound comprises any one or a combination of polyether, an alkyl ether, a glycol, a polyol or a compound having a C—O—C linkage.

24. The method as claimed in claim 16 wherein the material is fibre-based, sliver, roving and/or a non-woven material or the material is a woven or knitted material.

25. A wound dressing comprising the material as claimed in claim 1.

26. The material as claimed in claim 1 further defined as any one of the following:
a nasal packing material;
a dental packing material;
a suture; or
a seton.
